(19) United States
(12) Patent Application Publication
(10) Pub. No.: US 2008/0206885 A1
(43) Pub. Date: Aug. 28, 2008

(54) Environmentally sensitive fluorophores

(75) Inventors: Barbara Imperiali, Cambridge, MA (US); Galen S. Loving, Cambridge, MA (US)

Correspondence Address: Timothy Oyer;Wolf, Greenfield & Sacks, Federal Reserve Plaza, 600 Atlantic Avenue, Boston, MA, 02210-2206, US

(73) Assignee: Massachusetts Institute of Technology

(21) Appl. No.: 11/710,789

(22) Filed: Feb. 26, 2007

(51) Int. Cl. C07D 221/06; C07K 14/00; C07K 2/00; C07K 4/00; G01N 21/64

(52) U.S. Cl. 436/172; 530/300; 530/345; 546/99

(57) ABSTRACT

The present invention provides novel fluorophore compounds.

### FIELD OF THE INVENTION

The present invention relates to environment-sensitive fluorophores for reporting protein/protein and peptide/protein interactions.

### BACKGROUND OF THE INVENTION

Fluorescence is the result of a three-stage process that occurs when certain molecules absorb energy. The three stages comprise: 1) excitation; 2) excited-state lifetime; and 3) fluorescence emission. During stage 1, excitation, a photon of a certain energy is absorbed by the fluorophore. The fluorophore is initially in its ground state (S₀). Absorption of the photon causes that fluorophore to become excited. The energy of the absorbed photon is transferred to an electron. The electron is transferred to a higher energy state. The fluorophore exists in an excited electronic singlet state (S_{1′}), also called an excited state. The excited state of the fluorophore exists for a finite time, typically 10⁻⁸ to 10⁻⁹ seconds. During the excited state, the fluorophore changes in its translational, vibrational, and electronic energy states, and is subject to interactions with its molecular environment. The excited fluorophore releases energy and returns to the ground state, S₀, by fluorescence emission. Other processes such as fluorescence energy transfer, intersystem crossing, and collisional quenching may also depopulate S₁. The ratio of the number of fluorescence photons emitted, during the emission stage, to the number of photons absorbed, during the excitation stage, is termed the quantum yield. The quantum yield is a measure of the efficiency of fluorescence in competition with other processes such as fluorescence energy transfer, intersystem crossing, and collisional quenching.

During the third stage, fluorescence emission, a photon of energy hv (where h is Planck's constant and v is the frequency of the photon) is emitted, returning the fluorophore to its ground state S₀. The energy of the emitted photon is lower than the energy of the photon absorbed during the excitation stage. The difference in energy can be attributed to dissipation through processes during the excited-state lifetime, such processes include fluorescence energy transfer, intersystem crossing, and collisional quenching. The difference in energy of the absorbed photon and the emitted photon is called the Stokes shift. The Stokes shift is fundamental to the sensitivity of fluorescence techniques because it allows emission photons to be detected against a low background, and at a different wavelength than the excitation photons.

Compounds that have fluorescent properties have numerous uses. Fluorescent molecules can be used in single molecule spectroscopy, liquid crystal displays, light emitting diodes, solar energy collectors, and laser active media. Fluorescent molecules whose spectra or quantum yields are sensitive to their environments are valuable as fluorescent dyes and in the study of heterogeneous media, organized media, and biological media.

Environment-sensitive fluorophores are a special class of chromophores that have spectroscopic behavior that is dependent on the physicochemical properties of the surrounding environment. Solvatochromic fluorophores display sensitivity to the polarity of the local environment. These molecules exhibit a low quantum yield in aqueous solution, but become highly fluorescent in nonpolar solvents or when bound to hydrophobic sites in proteins or membranes. Examples of solvatochromic fluorophores include 2-propionyl-6-dimethylaminonaphthalene (PRODAN) (Weber et al. Biochemistry 1979, 18, 3075-3078; Cohen et al. Science 2002, 296, 1700-1703), 4-dimethylamino phthalimide (4-DMAP) (Saroja et al. J. Fluoresc. 1998, 8, 405-410), and 4-amino-1,8-naphthalimide derivatives (Grabchev et al. J. Photochem. Photobiol., A 2003, 158, 37-43; Martin et al. J. Lumin. 1996, 68, 157-146). Although PRODAN and its derivatives are widely used, these probes have limitations resulting from the relatively intense fluorescence even in aqueous environments. Thus, there is a need for alternate compounds.

U.S. Patent Application Publication No. 2006/0234206 discloses a 6-dimethylaminonaphthalimide group as an environment-sensitive fluorophore for reporting protein/protein and peptide/protein interactions. The fluorophore is integrated as part of an amino acid termed Dap(6-DMN). U.S. Patent Application Publication No. 2006/0205760, to Hartsel et al., discloses a naphthalimide compound with a monosubstituted amino group at the 4 position. Hartsel does not disclose an amino acid motif as an imide substitutent. The fluorophore compounds disclosed in the present invention have improved photophysical properties as an environment-sensitive reporter and improved chemical stability for biomolecule labeling and the detection of protein/protein and peptide/protein interactions.

### SUMMARY OF THE INVENTION

The present invention provides novel compounds of the formula (I):

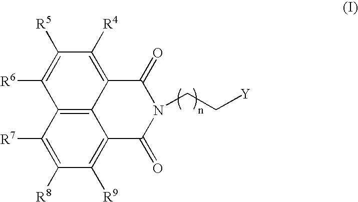

wherein R⁴, R⁵, R⁶, R⁷, R⁸, R⁹, n, and Y are as defined below. One preferred compound (VI) referred to as Dap(4-DMN) is disclosed.

The present invention also provides peptides containing the compound (I) of the present invention.

The present invention also provides a method for probing biological interactions using peptides containing the compound (I).

### BRIEF DESCRIPTION OF THE FIGURES

FIG. 1 illustrates the structures of the Dap(6-DMN) and Dap(4-DMN), the fluorescence excitation and emission maxima, and the changes in fluorescence spectra in methanol dioxane for the two compounds.

FIG. 2 illustrates the methods by which the fluorophore is incorporated into compounds, such as 3 and 4, that can be used for the selective chemical modification of cysteine in intact peptides and proteins.

### DESCRIPTION OF THE INVENTION

The present invention is directed to compounds and salts thereof, compostions and methods useful in monitoring biological interactions continuously with sensitive fluorophores that have readout. The compounds of the present invention are environment-sensitive fluorophores that have spectroscopic behavior that is dependent on the physicochemical properties of the surrounding environment. The compounds of the present invention can be used in biochemical research to monitor ions, small molecules, and biological processes such as protein folding, protein-protein interactions and phosphorylation events.

### Definitions

When describing the compounds, compositions, methods and processes of this invention, the following terms have the following meanings, unless otherwise indicated.

The term “hydroxy” means the —OH group.

The term “amino” means the —NR′R″ group, where R′ and R″ are each independently hydrogen or alkyl.

The term “thiol” means the —SR′ group, where R′ is hydrogen.

The term “halogen” or “halo” means a chlorine, bromine, iodine, or fluorine atom.

The term “alkyl” means a hydrocarbon group that may be linear, cyclic, or branched or a combination thereof having the number of carbon atoms designated (i.e., C1-8 means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc. Alkyl groups can be substituted or unsubstituted, unless otherwise indicated. Examples of substituted alkyl groups include haloalkyl, thioalkyl, aminoalkyl, and the like.

The term “aryl” means a polyunsaturated, aromatic hydrocarbon group having a single ring (monocyclic) or multiple rings (bicyclic or polycyclic), which can be fused together or linked covalently. Examples of aryl groups include phenyl and naphthalene-1-yl, naphthalene-2-yl, biphenyl and the like. Aryl groups can be substituted or unsubstituted, unless otherwise indicated.

The term “heteroaryl” means an aromatic group containing at least one heteroatom, where the heteroaryl group may be monocyclic or bicyclic. Examples include pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, quinolyl, isoquinolyl, pyrazolyl, indazolyl, pteridinyl, imidazolyl, triazolyl, tetrazolyl, oxazolyl, oxadiazolyl, thiadiazolyl, pyrrolyl, thiazolyl, furyl or thienyl.

The term “heterocyclyl” or “heterocyclic”, which are synonymous as used herein, means a saturated or unsaturated ring containing at least one heteroatom (typically 1 to 5 heteroatoms) selected from nitrogen, oxygen or sulfur. The heterocyclyl ring may be monocyclic or bicyclic. Examples of heterocycle groups include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene, quinuclidine and the

The term “ring” means a compound whose atoms are arranged in formulas in a cyclic form. The ring compound can be either carbocyclic or heterocyclic.

The term “carbocyclic” means a ring composed exclusively of carbon atoms.

The term “substituent” means an atom or a group that replaces another atoms or group in a molecule.

The term “N-terminal protecting group” refers to a group that prevents undesirable reaction of the amino functional group during subsequent transformations. The use of N-protecting groups is well known in the art for protecting groups against undesirable reactions during a synthetic procedure and many such protecting groups are know. Commonly used N-protecting groups are known to those skilled in the art, examples of which are disclosed in Greene and Wuts, Protective Groups in Organic Synthesis, 2nd ed.; John Wiley & Sons, New York, 1991). Examples of N-protecting groups include, but are not limited to, benzyl, substituted benzyl, benzyloxycarbonyl (Cbz), tert-butoxycarbonyl (Boc), 9-fluorenylmethoxycarbonyl (Fmoc), trityl, N-veratyloxycarbonyl (N-Voc), N-allyloxycarbonyl (N-Alloc) and N-pentenoyl (N-Pent), acyl groups including formyl, acetyl (Ac), trifluoroacetyl, trichloroacetyl, propionyl, pivaloyl, t-butylacetyl, acylisothiocyanate, aminocaproyl, benzoyl and the like; acyloxy groups, including t-butyloxycarbonyl (Boc), benzyloxycarbonyl (Cbz), 9-fluorenylmethoxycarbonyl (Fmoc), p-methoxybenzyloxycarbonyl, methoxycarbonyl, ethoxycarbonyl, allyloxycarbonyl and the like; sulfonyl groups such as benzenesulfonyl, p-toluenesulfonyl and the like; arylalkyl groups such as benzyl, triphenylmethyl, benzyloxymethyl and the like; and silyl groups such as trimethylsilyl, t-butyidimethylsilyl and the like.

The term “C-terminal protecting group” refers to a group that prevents undesirable reaction of the carboxyl functional group and includes, but is not limited to, C1-12 alkyl (e.g., tert-butyl) and C1-12 haloalkyl.

The term “chelation-enhanced fluorescence (CHEF)” means fluorescence enhancement of a compound as a result of metal ion binding (chelation) to that compound.

The term “capping group” means a chemical group connected to the N— or C-terminus of a peptide to prevent the peptide from degrading.

“Alkoxy” refers to —O-alkyl. Examples of an alkoxy group include methoxy, n-propoxy, etc.

“Haloalkyl”, as a substituted alkyl group, refers to a monohaloalkyl or polyhaloalkyl group, most typically substituted with from 1-3 halogen atoms. Examples include 1-chloroethyl, 3-bromopropyl, trifluoromethyl and the like.

All of the above terms (e.g., “alkyl,” “aryl,” “heteroaryl” etc.), in some embodiments, include both substituted and unsubstituted forms of the indicated groups. These groups may be substituted multiple times, as chemically allowed. Suitable substituents include alkyl, aryl, heteroaryl, heterocyclyl, halogen, alkoxy, oxygen, and

Certain compounds of the present invention can exist in unsolvated forms as well as solvated forms, including hydrated forms. In general, both solvated forms and unsolved forms are intended to be encompassed within the scope of the present invention. Certain compounds of the present invention may exist in multiple crystalline or amorphous forms (i.e., as polymorphs). In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are intended to be encompassed within the scope of the present invention.

“Fluorescence” encompasses the release of fluorescent energy. Less broadly, the term “fluorescence” refers to fluorescent emission, the rate of change of fluorescence over time (i.e., fluorescence lifetime), fluorescence polarization, fluorescence anisotropy, and fluorescence resonance energy transfer. See Eftink, M. R., Biophysical J. 66:482-501 (1994).

“Fluorescence probe molecule” refers to a compound of the present invention. The compound, after excitement by light of a defined wavelength or defined range of wavelengths, is capable of emitting fluorescent energy. The fluorescent molecule or a compound may be capable of binding to a peptide, protein, membrane or receptor.

The term “biological interactions” encompasses the interaction of a compound or molecule with a target molecule.

“Protein” and “peptide”, as used herein, are synonymous. For proteins or peptides, the term “unfolding” encompasses any change in structure due to heating. For example, the term “unfolding” refers to the transition of from the liquid crystalline state to the molten globule state. In the molten globule state, tertiary and quaternary structure has been altered, relative to the native state of the protein, and at least some secondary structure remains intact. The term “unfolding” also encompasses loss of crystalline ordering of amino acid side-chains, secondary, tertiary or quaternary structure. The term “unfolding” also encompasses formation of a random coil.

“Folding” and “refolding,” and “renaturing” refer to the acquisition of the correct amino acid side-chain ordering, secondary, tertiary, or quaternary structure, of a protein or a nucleic acid, which affords the full chemical and biological function of the biomolecule.

The term “target molecule” encompasses peptides, proteins, nucleic acids, ions, and other receptors. The term encompasses both enzymes, and proteins which are not enzymes. The term encompasses monomeric and multimeric proteins. Multimeric proteins may be homomeric or heteromeric. The term encompasses nucleic acids comprising at least two nucleotides, such as oligonucleotides. Nucleic acids can be single-stranded, double-stranded, or triple-stranded. The term encompasses a nucleic acid which is a synthetic oligonucleotide, a portion of a recombinant DNA molecule, or a portion of chromosomal DNA. The term target molecule also encompasses portions of peptides, proteins, and other receptors which are capable of acquiring secondary, tertiary, or quaternary structure through folding, coiling or twisting. The target molecule may be substituted with substituents including, but not limited to, cofactors, coenzymes, prosthetic groups, lipids, oligosaccharides, or phosphate groups.

The terms “target molecule” and “receptor” are synonymous.

Examples of target molecules are included, but not limited to those disclosed in Faisst, S. et al., Nucleic Acids Research 20:3-26 (1992); Pimentel, E., Handbook of Growth Factors, Volumes I-III, CRC Press, (1994); Gilman, A. G. et al., The Pharmacological Basis of Therapeutics, Pergamon Press (1990); Lewin, B., Genes V, Oxford University Press (1994); Roitt, I., Essential Immunology, Blackwell Scientific Publ. (1994); Shimizu, Y., Lymphocyte Adhesion Molecules, R G Landes (1993); Hyams, J. S. et al., Microtubules, Wiley-Liss (1995); Montreuil, J. et al., Glycoproteins, Elsevier (1995); Woolley, P., Lipases: Their Structure Biochemistry and Applications, Cambridge University Press (1994); Kurjan, J., Signal Transduction: Prokaryotic and Simple Eukaryotic Systems, Academic Press (1993); Kreis, T., et al., Guide Book to the Extra Cellular Matrix and Adhesion Proteins, Oxford University Press (1993); Schlesinger, M. J., Lipid Modifications of Proteins, CRC Press (1992); Conn, P. M., Receptors: Model Systems and Specific Receptors, Oxford University Press (1993); Lauffenberger, D. A. et al, Receptors. Models For Binding Trafficking and Signaling, Oxford University Press (1993); Webb, E. C., Enzyme Nomenclature, Academic Press (1992); Parker, M. G., Nuclear Hormone Receptors; Molecular Mechanisms, Cellular Functions Clinical Abnormalities, Academic Press Ltd. (1991); Woodgett, J. R., Protein Kinases, Oxford University Press (1995); Balch, W. E. et al., Methods in Enzymology, Vol. 257, Pt. C: “Small GTPases and Their Regulators: Proteins Involved in Transport,” Academic Press (1995); The Chaperonins, Academic Press (1996); Pelech, L., Protein Kinase Circuitry in Cell Cycle Control, R G Landes (1996); Atkinson, Regulatory Proteins of the Complement System, Franklin Press (1992); Cooke, D. T. et al., Transport and Receptor Proteins of Plant Membranes: Molecular Structure and Function, Plenum Press (1992); Schumaker, V. N., Advances in Protein Chemistry: Lipoproteins, Apolipoproteins, and Lipases, Academic Press (1994); Brann, M., Molecular Biology of G-Protein-Coupled Receptors: Applications of Molecular Genetics to Pharmacology, Birkhauser (1992); Konig, W., Peptide and Protein Hormones: Structure, Regulations, Activity—A Reference Manual, VCH Publ. (1992); Tuboi, S. et al., Post-Translational Modification of Proteins, CRC Press (1992); Heilmeyer, L. M., Cellular Regulation by Protein Phosphorylation, Springer-Verlag (1991); Takada, Y., Integrin: The Biological Problem, CRC Press (1994); Ludlow, J. W., Tumor Suppressors: Involvement in Human Disease, Viral Protein Interactions, and Growth Regulation, R G Landes (1994); Schlesinger, M. J., Lipid Modification of Proteins, CRC Press (1992); Nitsch, R. M., Alzheimer's Disease. Amyloid Precursor Proteins, Signal Transduction, and Neuronal Transplantation, New York Academy of Sciences (1993); Cochrane, C. G., et al., Cellular and Molecular Mechanisms of Inflammation, Vol. 3: Signal Transduction in Inflammatory Cells, Part A, Academic Press (1992); Gupta, S. et al., Mechanisms of Lymphocyte Activation and Immune Regulation IV: Cellular Communications, Plenum Press (1992); Authi, K. S. et al., Mechanisms of Platelet Activation and Control, Plenum Press (1994); Grunicke, H., Signal Transduction Mechanisms in Cancer, R G Landes (1995); Latchman, D. S., Eukaryotic Transcription Factors, Academic Press (1995).

The term “contacting a target molecule” refers broadly to placing the target molecule in solution with the molecule to be screened for binding or with the condition(s) to be tested for stabilizing the target molecule. Less broadly, contacting refers to the turning, swirling, shaking or vibrating of a solution of the target molecule and the molecule to be screened for binding. More specifically, contacting refers to the mixing of the target molecule with the molecule to be tested for binding. Mixing can be accomplished, for example, by repeated uptake and discharge through a pipette tip, either manually or using an automated pipetting device. Preferably, contacting refers to the equilibration of binding between the target molecule and the molecule to be tested for binding. Contacting can occur in the container, infra, or before the target molecule and the molecule to be screened are placed in the container.

The target molecule may be contacted with a nucleic acid prior to being contacted with the molecule to be screened for binding. The target molecule may be complexed with a peptide prior to being contacted with the molecule to be screened for binding. The target molecule may be phosphorylated or dephosphorylated prior to being contacted with the molecule to be screened for binding.

A carbohydrate moiety may be added to the target molecule before the target molecule is contacted with the molecule to be screened for binding. Alternatively, a carbohydrate moiety may be removed from the target molecule before the target molecule is contacted with the molecule to be screened for binding.

The term “container” refers to any vessel or chamber in which the receptor and molecule to be tested for binding can be placed. The term “container” encompasses reaction tubes (e.g., test tubes, microtubes, vials, etc.).

The term “biological sample” refers to the contents of a container.

“Spectral emission,” “thermal change,” and “physical change” encompass the release of energy in the form of light or heat, the absorption of energy in the form or light or heat, changes in turbidity and changes in the polar properties of light. Specifically, the terms refer to fluorescent emission, fluorescent energy transfer, absorption of ultraviolet or visible light, changes in the polarization properties of light, changes in the polarization properties of fluorescent emission, changes in the rate of change of fluorescence over time (i.e., fluorescence lifetime), changes in fluorescence anisotropy, changes in fluorescence resonance energy transfer, changes in turbidity, and changes in enzyme activity. Preferably, the terms refer to fluorescence, and more preferably to fluorescence emission. Fluorescence emission can be intrinsic to a protein or can be due to a fluorescence reporter molecule. The use of fluorescence techniques to monitor protein unfolding is well known to those of ordinary skill in the art. For example, see Eftink, M. R., Biophysical J. 66:482-501 (1994).

“Biochemical conditions” encompass any component of a physical, chemical, or biochemical reaction. Specifically, the term refers to conditions of temperature, pressure, protein concentration, pH, ionic strength, salt concentration, time, electric current, potential difference, concentrations of cofactor, coenzyme, oxidizing agents, reducing agents, detergents, metal ion, ligands, or glycerol.

### Compounds

The compounds of the present invention undergo enhanced fluorescence in nonpolar environments as compared to polar environments. Examples of nonpolar environments include nonpolar solvents and hydrophobic proteins or membranes.

In one embodiment, the compound of the present invention is of the formula (I):

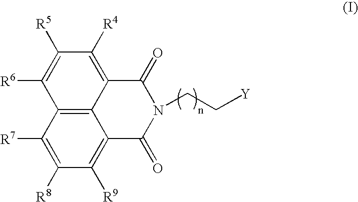

wherein R⁴, R⁵, R⁶, R⁷, R⁸, and R⁹ are each independently hydrogen, halogen, or alkyl, wherein at least one of R⁴, R⁵, R⁶, R⁷, R⁸, and R⁹ is —NR¹R², —OH, —SH, —OR¹, or —SR¹;

R¹ and R² are each independently substituted or unsubstituted alkyl, or R¹ and R² together with the nitrogen to which they are attached, form a substituted or unsubstituted 5- or 6-membered ring;

Y is halogen, —SH, —NHR³, —C(O)X, -maleimidyl, —NHCOR³, or —CH(NHR³)COOH;

R³ is hydrogen, substituted or unsubstituted alkyl, or an N-protecting group;

X is hydrogen, halogen, hydroxy or alkoxy; and

n is 0, 1, 2, or 3.

Preferably, one of R⁶ and R⁷ is —NR¹R². Preferably, R¹ or R² is alkyl. More preferably, both R¹ and R² are alkyl. Even more preferably, both R¹ and R² are methyl, ethyl or propyl. Preferably, R¹ and R² together with the nitrogen to which they are attached are pyrrolidinyl, piperdinyl, or morpholinyl. Preferably, R³ is an N-protecting group. More preferably, R³ is Boc, Cbz, or Fmoc. Even more preferably, R³ is Fmoc. The compound can be a D-isomer or an L-isomer. Preferably, the compound is the D-isomer.

In a preferred embodiment, the compound of the present invention is of the formula (II):

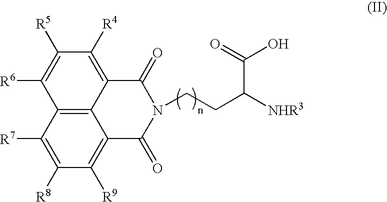

wherein n, R³, R⁴, R⁵, R⁶, R⁷, R⁸, and R⁹ are as described above for formula (I).

In another embodiment, the compound of the present invention is of the formula (III):

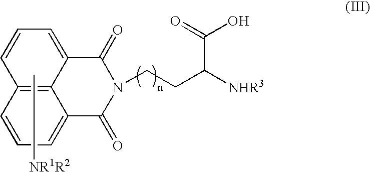

wherein —NR¹R² can substitute any open valence of any ring within structure (III); and n, R¹, R² and R³ are as described above for formula (I).

In another embodiment, the compound of the present invention is of the formula (IV):

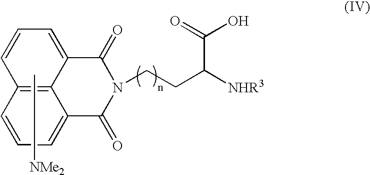

wherein —NMe₂ can substitute any open valence of any ring within structure (IV); and n and R³ are as described above for formula (I).

In a preferred embodiment, the compound of the present invention is of the formula (V):

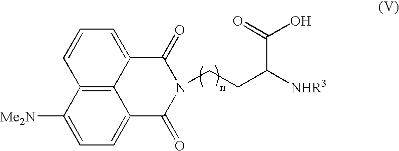

wherein n and R³ are as described above for formula (I).

In another preferred embodiment, the compound of the present invention is of the formula (VI):

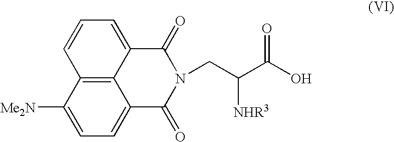

wherein R³ is as described above for formula (I).

In another embodiment, the compound of the present invention is of the formula (VII):

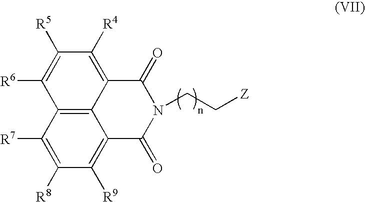

wherein Z is halogen, —SH, —NHR³, —C(O)X, -maleimidyl, or —NHCOR³; and n, R⁴, R⁵, R⁶, R⁷, R⁸, and R⁹ are as described above for formula (I). Preferably, Z is halogen, —SH, or —NH₂.

In a preferred embodiment, the compound of the present invention is of the formula (VIII):

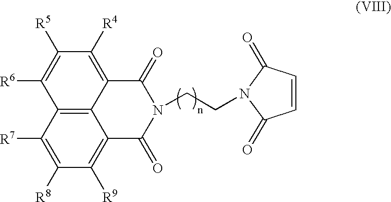

wherein n, R⁴, R⁵, R⁶, R⁷, R⁸, and R⁹ are as described above for formula (I).

In another preferred embodiment, the compound of the present invention is of the formula (IX):

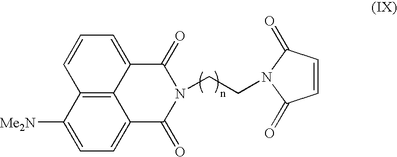

wherein n is as described above for formula (I).

In a preferred embodiment, the compound of the present invention is of the formula (X):

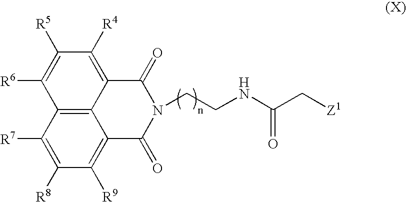

wherein Z¹ is halogen; and n, R⁴, R⁵, R⁶, R⁷, R⁸, and R⁹ are as described above for formula (I).

In another preferred embodiment, the compound of the present invention is of the formula (XI):

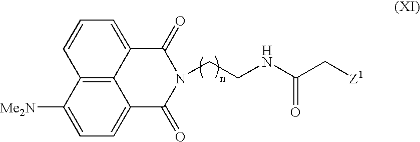

wherein Z¹ and n are as described above for formula (X).

In one embodiment, the compound of the present invention is of the formula (XII):

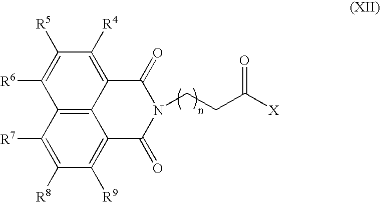

wherein n, X, R⁴, R⁵, R⁶, R⁷, R⁸, and R⁹ are as described above for formula (I).

### Peptides

The fluorophore compounds, such as formulae (II) and (VI), can be formed into peptides using standard peptide synthesis (solid phase or solution phase). Standard peptide synthesis is well-known in the art. See, for example, Fmoc Solid Phase Peptide Synthesis—A Practical Approach, Oxford University Press, 2003, Eds W. C. Chan and P. D. White (ISBN 0 19 963 724 5); and The Chemical Synthesis of Peptides, Clarendon Press, Oxford, 1994, Jones, J. (ISBN 0 19 855839 2).

When the fluorophore compounds are coupled to peptide after synthesis, the peptide is first synthesized and protecting groups on the side chains of the peptide are selectively removed. Then the fluorophore compounds, such as formulae (VII), (IX) and (XI), can be coupled to the side chains of formed peptides using standard coupling methods. For example, when Y is a maleimidyl or an alpha-halo-amide, the compound can be coupled to a residue containing a thiol group in its side chain (such as Cys). Alternatively, when Y is halogen, the compound can be coupled to a residue containing a thiol group in its side chain (such as Cys), forming a thioether linkage. Alternatively, when Y is an amine, it can be coupled to a residue containing a carboxylic acid in its side chain (such as Asp or Glu), forming an amide linkage. Alternatively, when Y is thiol, it can be coupled to a residue containing a thiol group in its side chain (such as Cys), forming a disulfide linkage. Alternatively, when Y is a carboxylic acid, it can be coupled to a residue containing an amine in its side chain. Alternatively, when Y is an aldehyde, it can be coupled to a residue containing amine via reductive amination. The fluorophore-containing peptide is then deprotected and purified.

Selective deprotection of amino acids is well known in the art. A preferred method is to use orthogonal side-chain protection such as allyl (OAll) (for the carboxyl group in the side chain of glutamic acid or aspartic acid, for example), allyloxy carbonyl (Alloc) (for the amino nitrogen in the side chain of lysine or ornithine, for example), p-methoxytrityl (MMT) or acetamidomethyl (Acm) (for the sulfhydryl of cysteine). OAll and Alloc are easily removed by Pd, Acm is easily removed by iodine treatment, and MMT is easily removed by very mild acid treatment.

Methods for introduction and removal of N-protecting groups are known to those skilled in the art, examples of which are disclosed in Greene and Wuts, Protective Groups in Organic Synthesis, 2nd ed.; John Wiley & Sons, New York, 1991.

In one embodiment, the peptide of the present invention comprises an amino acid residue, wherein the side chain of the amino acid residue is modified with a compound of the formula (VII):

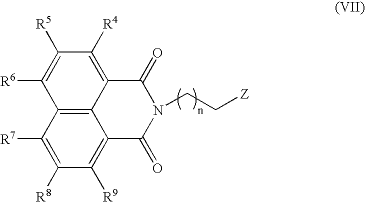

wherein R⁴, R⁵, R⁶, R⁷, R⁸, and R⁹ are each independently hydrogen, halogen, or alkyl, wherein at least one of R⁴, R⁵, R⁶, R⁷, R⁸, and R⁹ is —NR¹R², —OH, —SH, —OR¹, or —SR¹;

R¹ and R² are each independently substituted or unsubstituted alkyl, or R¹ and R² together with the nitrogen to which they are attached, form a substituted or unsubstituted 5- or 6-membered ring;

Z is halogen, —SH, —NHR³, —C(O)X, -maleimidyl, or —NHCOR³;

R³ is hydrogen, substituted or unsubstituted alkyl, or an N-protecting group;

X is hydrogen, halogen, hydroxy or alkoxy; and

n is 0, 1, 2, or 3.

Preferably, the amino acid that is modified is cysteine, aspartic acid, glutamic acid, or lysine.

In another embodiment, the peptide of the present invention comprises an amino acid residue of the formula (XIII):

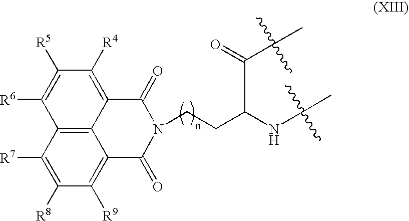

wherein n, R⁴, R⁵, R⁶, R⁷, R⁸, and R⁹ are as described above for formula (VII).

In a preferred embodiment, the peptide of the present invention comprises an amino acid residue of the formula (XIV):

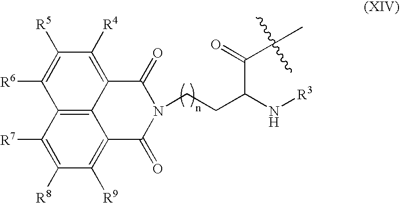

wherein R³ is an N-protecting group; and n, R⁴, R⁵, R⁶, R⁷, R⁸, and R⁹ are as described above for formula (VII).

In another preferred embodiment, the peptide of the present invention comprise an amino acid residue of the formula (XV):

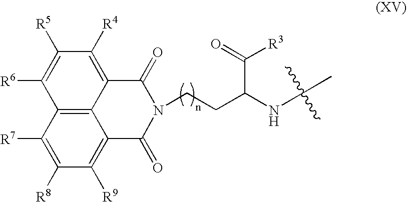

wherein in R³ is a C-protecting group; and n, R⁴, R⁵, R⁶, R⁷, R⁸, and R⁹ are as described above for formula (VII).

The amino acid residue of the present invention can be the D-isomer or the L-isomer. Preferably, the amino acid residue is the D-isomer. Preferably, the peptide of the present invention comprises 2-100 amino acids. More preferably, the peptide comprises 2-30 amino acids. Further more preferably, the peptide comprises 2-20 amino acids. Ever more preferably, the peptide comprises 3-10 amino acids.

Preferably, the peptide of the present invention further comprises a target recognition sequence. More preferably, the peptide comprises an SH2-domain recognition sequence. Even more preferably, the SH2-domain recognition sequence is pTyr-Asp-His-Pro or pTyr-Glu-Asn-Val.

In one embodiment of the present invention, a method of probing biological interactions comprises (a) preparing a peptide according to one of the embodiments of the present invention; (b) contacting a target molecule with the peptide to form a biological sample; and (c) monitoring the fluorescence of the biological sample.

### Use of the Compounds and Peptides

Compounds of the present invention are useful as fluorescence probe molecules in applications wherein fluorescence probes are known to be useful. In using a compound of the present invention as a fluorescence probe molecule, the compound is added to a sample to be probed. The sample comprising the compound is then exposed to a light source. The light source produces light that is limited to a range of wavelengths. The range of wavelengths is between about 320 and about 530 nanometers (nm), preferably between about 380 and about 490 nm, most preferably between about 440 and about 445 nm. Upon exposure to a light source, the compound of the present invention is fluorescent and emits fluorescent energy. The emitted fluorescent energy is detected using methods well known in the art. The intensity and wavelength of the emitted fluorescent energy provides information about the sample. The emitted fluorescent energy preferably has a range of wavelengths between about 410 and about 630 nm, preferably between about 490 and about 542 nm.

The fluorescence of a molecule is defined by the quantum yield. The quantum yield is the ratio of the photons absorbed by the compound to the photons emitted through fluorescence by the compound. Compounds of the present invention have quantum yields that are preferably low in aqueous solutions and high in non-polar environments. Quantum yields range from about 0.001 and about 0.1, preferably between about 0.001 and about 0.005 for aqueous solutions, preferably between about 0.2 and about 0.7 for non-polar environment.

Fluorescence can also be evaluated by determining the dipole moment change between the ground and excited state. The change in the dipole moment can be estimated from a plot of the Stokes shift vs. the orientation polarizability, known as a Lippert-Mataga plot (Lippert, V. E. Z. Elektrochem. 1957, 61, 962-975; Mataga, N.; Kaifu, Y.; Koizumi, M. Bull. Chem. Soc. Jpn. 1956, 29, 465-470).

Fluorescence is sensitive to the pH of the surrounding environment. Compounds of the present invention are useful as fluorescence probes in the pH range from about 4 to about 8.

Compounds of the present invention are useful in monitoring biological interactions. Biological interactions play important roles in the sequence and mechanisms of action of various cellular processes and signal pathways. The time course, nature, and sequence of the different cellular processes can be elucidated by in situ observation using the compounds of the present invention. Specific inhibitors and/or activators of the cellular processes and signal pathways being studied may optionally be used in addition to compounds of the present invention.

Biological interactions, as defined herein, comprise the interaction of a compound or molecule with a target molecule. Examples of target molecules include peptides, proteins, enzymes, nucleic acids, ions, and other receptors; preferably metal ion chelators, proteases, polymerases, hydrolases, phosphatases, and kinases; more preferably protein domains, and protein domains of phosphatases and kinases.

Proteins and protein-protein interactions play a central role in the various essential biochemical processes. For example, these interactions are evident in the interaction of hormones with their respective receptors, in the intracellular and extracellular signaling events mediated by proteins, in enzyme substrate interactions, in intracellular protein trafficking, in the formation of complex structures like ribosomes, viral coat proteins, and filaments, and in antigen-antibody interactions. These interactions are usually facilitated by the interaction of small regions within the proteins that can fold independently of the rest of the protein. These independent units are called protein domains. Abnormal or disease states can be the direct result of aberrant protein-protein interactions. Protein-protein interactions are also central to the mechanism of a virus recognizing its receptor on the cell surface as a prelude to infection. Identification of domains that interact with each other not only leads to a broader understanding of protein-protein interactions, but also aids in the design of inhibitors of these interactions.

Phosphorylation-dependent peptide-protein interactions include phosphoserine peptides with 14-3-3, which is a protein involved in cell cycle control (Muslin, A. J., Tanner, J. W., Allen, P. M., Shaw, A. S. Cell 1996, 84, 889-897), and phosphotyrosine peptides with SH2 domains. SH2 domains are binding modules that are involved in tyrosine kinase signaling networks and recognize phosphotyrosine-containing peptide sequences. The phosphotyrosine binding is complemented by simultaneous peptide-protein interactions on the protein surface. Examples of SH2 domains include Abl SH2, Crk SH2, and C-terminal P13K SH2 which can be expressed in bacteria as GST fusion proteins, which are referred to as GST-Abl SH2, GST-Crk SH2, and GST-PI3K SH2.

Recognition sequences for SH2 domains comprise a phosphotyrosine residue and other amino acids. The recognition sequence is different for different SH2 domains. Amino acid recognition sequences for binding members of the SH2 domain family are disclosed in Songyang, Z. et al.; Cell 1993, 72, 767-778. For the Crk SH2 domain, the recognition sequence is pTyr-Asp-His-Pro. For the Abl SH2 domain, the recognition sequence is pTyr-Glu-Asn-Val.

Compounds of the formula (I) are useful for studying the peptide-protein interactions on the protein surface of the SH2 domain. Compounds of formula (I) can be incorporated into peptides containing the desired SH2 recognition sequence. Table 1 shows peptides incorporating the Crk SH2 or Abl SH2 recognition sequences and Dap(4-DMN) into the (+2) position relative to the phosphotyrosine residue.

TABLE 1
Peptide Sequences and Corresponding SH2 Domain Target

| | Target | |
|---|---|---|
| Peptide | SH2 | Peptide sequence |
| Crk-bp | Crk | Ac-Glu-Dap(4-DMN)-Gln-pTyr-Asp-His-Pro-Asn-Ile- |
| | | (CONH₂) |
| Crk-bp2 | Crk | Ac-Glu-Dap(4-DMN)-Gly-pTyr-Asp-His-Pro-Asn-Ile- |
| | | (CONH₂) |
| Abl-bp | Abl | Ac-Glu-Dap(4-DMN)-Gly-pTyr-Glu-Asn-Val-Gln-Ser- |
| | | (CONH₂) |
| Abl-bp2 | Abl | Ac-Glu-Dap(4-DMN)-pTyr-Glu-Asn-Val-Gln-Ser- |
| | | (CONH₂) |

The peptides of Table 1 can be incubated with targeted and nontargeted SH2 domains. The binding of peptides Crk-bp, Crk-bp2, Abl-bp, and Abl-bp2 to SH2 target domains can be studied by fluorescence titration.

Compounds of the present invention are also useful in a method of monitoring biological interactions, comprising providing a compound of the present invention, contacting a target molecule with the compound to form a biological sample, and monitoring the fluorescence of the biological sample. Preferably, the compound is a peptide. More preferably, the compound is a peptide containing at least one of the formulae (II) and (VI). Alternatively, the compound is a peptide containing an amino acid residue that is modified by at least one of the formulae (VII), (IX) and (XI).

In the methods of the present invention, the monitoring step comprise contacting the compound with the one or more target molecules or different biochemical conditions, wherein the measuring step comprises exciting the compound of the present invention with light, and measuring the fluorescence.

In all methods of using a compound of the present invention, the concentration used will depend on the detection equipment. Typically, the concentration of the compound is from greater than about 0.1 nM.

The sensor of the present invention can be used in a method for detecting biological interactions. The method of the present invention comprises providing a peptide incorporating an amino acid of the formula (II), contacting a target molecule with the peptide to form a biological sample, and monitoring the fluorescence of the biological sample. Alternatively, the method of the present invention comprises providing a peptide comprising an amino acid residue that is modified by a compound of the formula (VII), contacting a target molecule with the peptide to form a biological sample, and monitoring the fluorescence of the biological sample.

### EXAMPLES

Synthesis of Compounds:

The synthesis of 4-DMN is similar to that of 6-DMN. See, for example, the synthesis method set forth in U.S. Pat. App. Pub. No. 2006/0234206, the entirety of which is herein incorporated by reference.

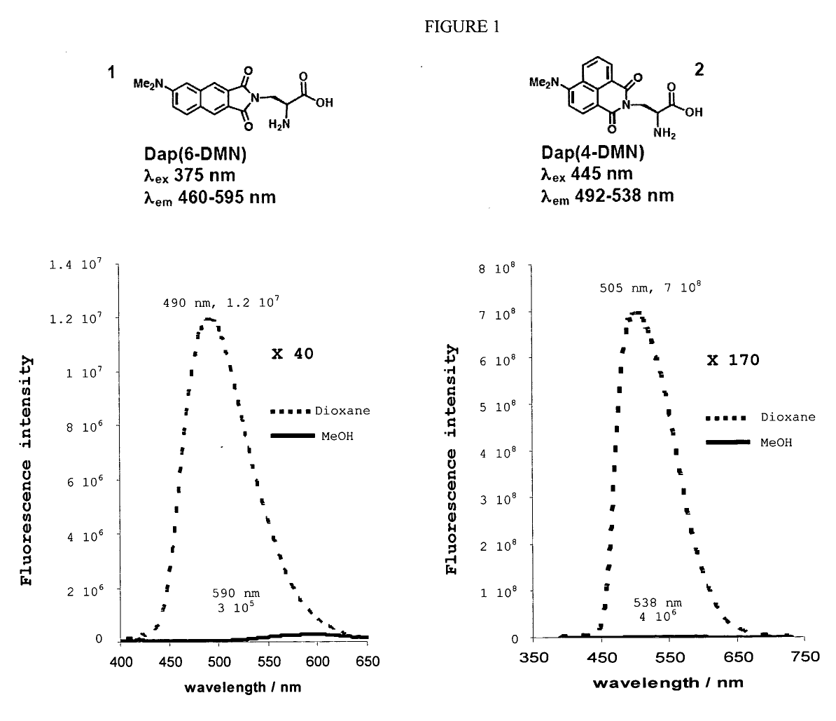

FIG. 1 illustrates the structures of the Dap(6-DMN) and Dap(4-DMN), the fluorescence excitation and emission maxima, and the changes in fluorescence spectra in methanol and dioxane for the two compounds. The latter are indicative of the large changes that can be achieved for signaling protein/protein and peptide/protein interactions when the 4-DMN group is integrated to one of the binding partners either as an amino acid (e.g. 2) or via chemical modification of a cysteine in one of the sequences. The fluorophore is incorporated into the amino acid (2) for integration into peptides by solid-phase peptide synthesis or into proteins by protein semi-synthesis.

Synthesis of the Peptides:

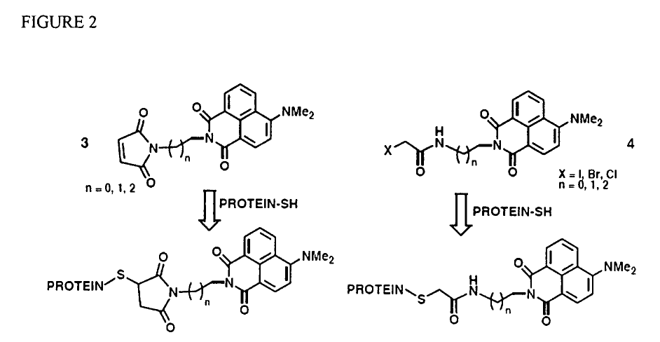

Referring to FIG. 2, the fluorophore is incorporated into compounds, such as 3 and 4 that can be used for the selective chemical modification of cysteine in intact peptides and proteins. The sensor peptides are synthesized via standard solid-phase peptide synthesis.

The peptide synthesis is carried out using standard Fmoc-based solid phase peptide synthesis (SPPS) protocols on a 0.05 to 0.1 mmol scale using a 0.21 mmol/g loading PAL-PEG-PS solid support. Amino acids are coupled in three-fold excess using a mixture of 0.2 M HBTU/0.2 M HOBt in DMF as activating agents. Each amino acid is activated for two minutes with the HBTU/HOBt mixture (1 eq.) and diisopropylethylamine (DIPEA), 0.195 M in DMF (1.5 eq.) before being added to the resin. Peptide coupling is monitored using the 2,4,6-trinitrobenzenesulphonic acid (TNBS) test (Hancock, W. S.; Battersby, J. E. Anal. Biochem. 1976, 71, 260-264). Amino acids are used as protected Fmoc-amino acids with the standard side chain protecting groups. High-performance liquid chromatography (HPLC) is performed using a Waters 600E HPLC fitted with a Waters 600 automated control module and a Waters 2487 dual wavelength absorbance detector recording at 228 and 280 nm. For analytical HPLC, a Beckman Ultrasphere C18, 5 □m, 4.6×150 mm reverse-phase column is used. For preparative separations, a YMC-pack, C18, 250×20 mm reversed phase column is used. The standard gradient for analytical and preparative HPLC used is 93:7 to 5:95 over 35 minutes (water:acetonitrile, 0.1% TFA). The DMN side chain proved resistant to the standard mildly basic amino acid coupling conditions (0.12 M diisopropylethylamine), the Fmoc deprotection conditions (20% piperidine), and the acidic resin cleavage and deprotection cocktail (95% TFA).

### Assays:

Methods for quantum yields measurement, Lippert-Mataga plots, fluorescence titrations and determination of binding constant (Kd), expression of GST-Crk SH2, western blot, expression of GST-AbI SH2, GST-Src SH2 and GST-PI3K SH2 are similar to the methods set forth in U.S. Pat. App. Pub. No. 2006/0234206, the entirety of which is herein incorporated by reference.

While the invention has been described with reference to certain embodiments, other features may be included without departing from the spirit and scope of the invention. It is therefore intended that the foregoing detailed description be regarded as illustrative rather than limiting, and that it be understood that it is the following claims, including all equivalents, that are intended to define the spirit and scope of this invention.

1. A compound of the formula (I): 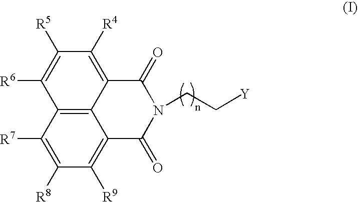
wherein R⁴, R⁵, R⁶, R⁷, R⁸, and R⁹ are each independently hydrogen, halogen, or alkyl, wherein at least one of R⁴, R⁵, R⁶, R⁷, R⁸, and R⁹ is —NR¹R², —OH, —SH, —OR¹, or —SR¹;
R¹ and R² are each independently substituted or unsubstituted alkyl, or R¹ and R² together with the nitrogen to which they are attached, form a substituted or unsubstituted 5- or 6-membered ring;
Y is halogen, —SH, —NHR³, —C(O)X, -maleimidyl, —NHCOR³, or —CH(NHR³)COOH;
R³ is hydrogen, substituted or unsubstituted alkyl, or an N-protecting group;
X is hydrogen, halogen, hydroxy or alkoxy; and
n is 0, 1, 2, or 3.

2. The compound of claim 1, which is of the formula (II):

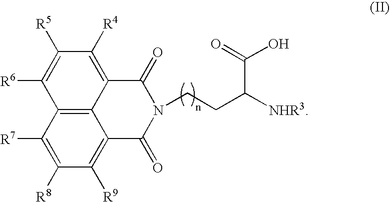

3. The compound of claim 2, which is of the formula (III): 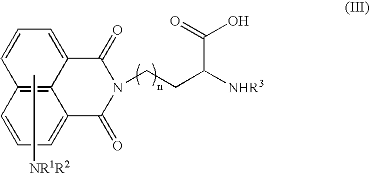
wherein —NR¹R² can substitute any open valence of any ring within structure (III).

4. The compound of claim 2, which is of the formula (IV): 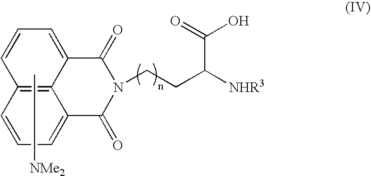
wherein —NMe₂ can substitute any open valence of any ring within structure (IV).

5. The compound of claim 2, which is of the formula (V):

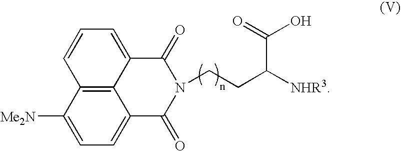

6. The compound of claim 2, which is of the formula (VI):

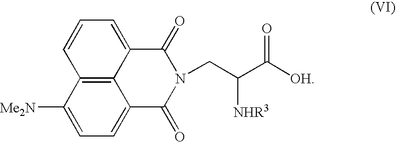

7. The compound of claim 1, which is of the formula (VII): 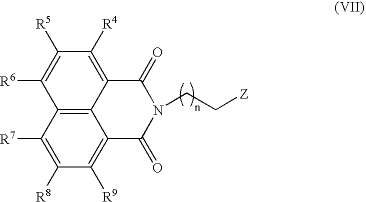
wherein Z is halogen, —SH, —NHR³, —C(O)X, -maleimidyl, or —NHCOR³.

8. The compound of claim 7, wherein Z is halogen, —SH, or —NH₂.

9. The compound of claim 7, which is of the formula (VIII):

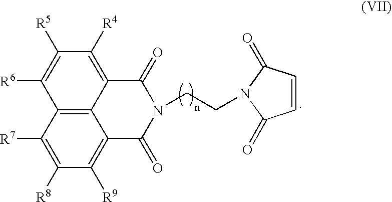

10. The compound of claim 9, which is of the formula (IX):

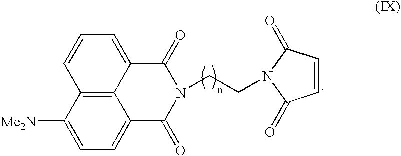

11. The compound of claim 7, which is of the formula (X): 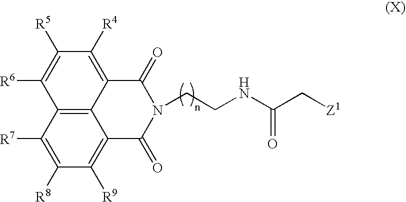
wherein Z¹ is halogen.

12. The compound of claim 11, which is of the formula (XI):

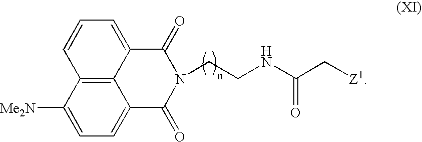

13. The compound of claim 7, which is of the formula (XII):

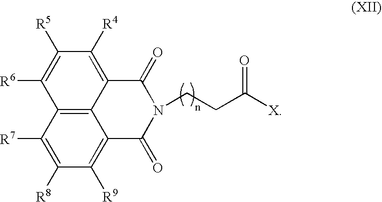

14. A peptide comprising an amino acid residue, wherein the side chain of the amino acid residue is modified with a compound of the formula (VII): 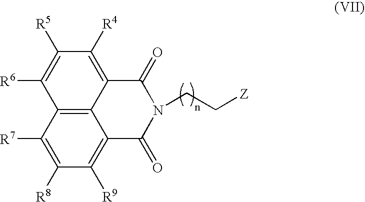
wherein R⁴, R⁵, R⁶, R⁷, R⁸, and R⁹ are each independently hydrogen, halogen, or alkyl, wherein at least one of R⁴, R⁵, R⁶, R⁷, R⁸, and R⁹ is —NR¹R², —OH, —SH, —OR¹, or —SR¹;
R¹ and R² are each independently substituted or unsubstituted alkyl, or R¹ and R² together with the nitrogen to which they are attached, form a substituted or unsubstituted 5- or 6-membered ring;
Z is halogen, —SH, —NHR³, —C(O)X, -maleimidyl, or —NHCOR³;
R³ is hydrogen, substituted or unsubstituted alkyl, or an N-protecting group;
X is hydrogen, halogen, hydroxy or alkoxy; and
n is 0, 1, 2, or 3.

15. The peptide of claim 14, wherein the amino acid that is modified is selected from the group consisting of cysteine, aspartic acid, glutamic acid, and lysine.

16. A peptide comprising an amino acid residue of the formula (XIII): 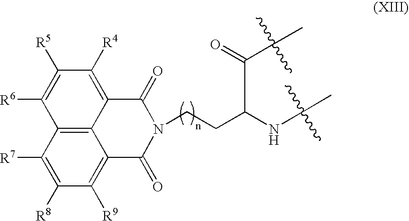
wherein R⁴, R⁵, R⁶, R⁷, R⁸, and R⁹ are each independently hydrogen, halogen, or alkyl, wherein at least one of R⁴, R⁵, R⁶, R⁷, R⁸, and R⁹ is —NR¹R², —OH, —SH, —OR¹, or —SR¹;
R¹ and R² are each independently substituted or unsubstituted alkyl, or R¹ and R² together with the nitrogen to which they are attached, form a substituted or unsubstituted 5- or 6-membered ring; and
n is 0, 1, 2, or 3.

17. The peptide of claim 16, wherein the amino acid residue is of the formula (XIV): 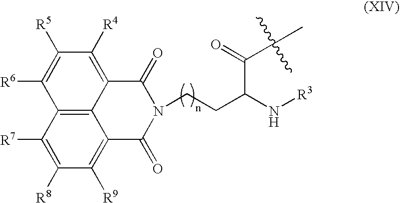
wherein R³ is an N-protecting group.

18. The peptide of claim 16, wherein the amino acid residue is of the formula (XV): 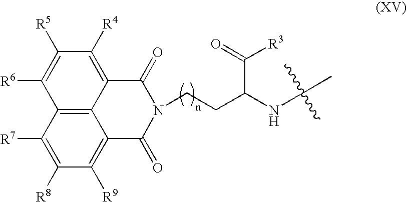
wherein R³ is a C-protecting group.

19. The peptide of claim 16, wherein the amino acid residue is the D-isomer.

20. The peptide of claim 16, wherein the peptide comprises 2-100 amino acids.

21. The peptide of claim 16, further comprising a target recognition sequence.

22. The peptide of claim 21, wherein the target recognition sequence comprises an SH2-domain recognition sequence.

23. The peptide of claim 22, wherein the SH2-domain recognition sequence comprises one of pTyr-Asp-His-Pro and pTyr-Glu-Asn-Val.

24. A method of probing biological interactions comprising:
(a) preparing the peptide of claim 14 or claim 16;
(b) contacting a target molecule with the peptide to form a biological sample; and
(c) monitoring the fluorescence of the biological sample.
